(19) United States
(12) Patent Application Publication
(10) Pub. No.: US 2007/0050866 A1
(43) Pub. Date: Mar. 1, 2007

(54) Control of hypocotyllength and flowering time by COL8 gene

(76) Inventors: Tomohiro Kiyosue, Kitagun (JP); Tomoyuki Takase, Kitagun (JP)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/512,468

(22) Filed: Aug. 30, 2006

Related U.S. Application Data
(60) Provisional application No. 60/712,948, filed on Aug. 31, 2005.

(51) Int. Cl. C07H 21/04; C12N 15/82; A01H 1/00; C07K 14/415; C12N 15/87; C12N 5/04

(52) U.S. Cl. 800/287; 536/23.6; 530/370; 435/419; 435/468; 800/294

(57) ABSTRACT

The present invention provides an isolated nucleic acid including a nucleotide sequence encoding COL8, which controls hypocotyl length and flowering time in a plant. It is found that plants over-expressing COL8 under long day conditions show slight hypocotyl elongation and delayed flowering in comparison with the wild type. In addition, COL8 and FKF1, which is a circadian-clock related gene, are found to be localized at the same site in a plant cell, indicating the existence of an interaction between the two.

### CROSS-REFERENCE TO RELATED APPLICATIONS

This application claims priority under 35 U.S.C. 119 based upon Provisional Patent Application No.60/712,948, filed on Aug. 31, 2005. The entire disclosure of the aforesaid application is incorporated herein by reference.

### BACKGROUND OF THE INVENTION

1. Field of the Invention

The present invention relates to the genetic control of flowering in plants and expression of genes involved therein. In particular, the present invention relates to a nucleic acid comprising a nucleotide sequence encoding COL8, which controls hypocotyl length and flowering time in a plant, and use of the gene in plants.

2. Description of the Related Art

Light, photoperiod, and temperature often act as important environmental cues for the initiation of flowering. Flowering time has been the important subject of classic breeding in agriculture. For example, for those plants in which the leaves or tubers are a commercial product, such as lettuce, spinach and so on, it is desirable to avoid “bolting” (initiation of flowering and stem elongation) at too early a stage. Another example is early variety of grain crops, which yields relatively a low amount of harvest due to its short growth time, but it is advantageous in that it can be on the market early. In general, delaying flowering is important in increasing the yield of plants-from which the roots or leaves are harvested. Furthermore, delayed flowering may be utilized such that pollen from a plant is not available at the same time that flowers on the same plant are receptive to the pollen, thereby eliminating the possibility of self-pollination.

However, there is very little information on the molecular mechanisms that directly regulate the developmental pathway from reception of the inductive light signals to the onset of flowering. It is known that at least part of the genetic hierarchy controlling flowering onset is responsive to the number of hours of light within a 24 hour light/dark cycle. Such photoperiodic responses have long been thought to be tied to biological clocks based on endogenous circadian rhythms. It has been suggested that under long day conditions, light signals perceived by the photoreceptors trigger the activation of a facultative long day pathway in which the “clock-related” genes, such as ELF3, TOC1, LHY, CCA1, FKF1, and ZTL, are involved (Blazquez, 2000).

Arabidopsis thaliana is a facultative long day plant, flowering early under long day conditions and late under short day conditions. Because it has a small well-characterized genome, is relatively easily transformed and regenerated and has a rapid growing cycle, Arabidopsis is an ideal model plant in which to study flowering and its control.

Meanwhile, the production of transgenic plants carrying a heterologous gene sequence has become one modem technique and is now routinely practiced by plant molecular biologists. Methods for incorporating an isolated gene sequence into an expression cassette, producing plant transformation vectors, and transforming many types of plants are now well known. Thus, a gene of interest can be routinely introduced into desired plants with practical aims to enhance commercial value, yields, and environmental adaptability. For example, flowering plants can be engineered so that they flower earlier or later than control plants without any detrimental phenotype effects.

With the background described as above, numerous studies have been carried out for the purpose of understanding the genetic mechanisms which influence flowering for altering the flowering characteristics of the target plant. Listed below are some examples.

U.S. Pat. No. 6,265,637 discloses a nucleic acid molecule comprising a nucleotide sequence encoding a polypeptide with LHY (Late Elongated Hypocotyl) function, which refers to the ability to influence the timing of flowering phenotypically like the LHY gene of Arabidopsis thaliana. The LHY gene is found to be one of the genes required for the response to photoperiod, and it is suggested that over-expression of LHY delays flowering under long days while under-expression promotes flowering in a transgenic plant.

U.S. Pat. No. 6,689,940 provides an isolated ELF3 (Early Flowering) gene from Arabidopsis that is shown to complement the ELF3 photoperiod-insensitive flowering and elongated hypocotyls defects when introduced into elf3 mutant plants.

In U.S. Pat. No. 6,727,407, it is shown that over-expression of CKB3 results in increased CK2 activity and in shorter periods of rhythmic expression of CCA1 (Circadian Clock Associated 1) and LHY, as well as of four other circadian clock-controlled genes. This leads to a significant shortening of time to flowering under short day conditions.

U.S. Pat. No. 6,586,252 discloses a nucleic acid molecule encoding the catalytic subunit of a protein phosphatase (PP2AC-JD) and methods for generating transgenic higher plants transformed with the nucleic acid molecule to engineer flowering time. The PP2AC-JD is shown to interact with the phytochrome A, a primary photoreceptor related to the photoperiodic control of flowering.

In U.S. Patent Application Publication No. 2005/0066393, it is shown that suppressing PHYC gene expression enables the promotion of plant flowering under long day conditions. The disclosed invention provides phyC mutants that hasten the onset of flowering and a method to accelerate flowering by suppressing the PHYC gene expression, in particular to produce a new early-harvesting rice cultivar.

U.S. Patent Application Publication No. 2005/0183166 provides a COG gene which controls the flowering time and a method for delaying the flowering time in plants by over-expressing the COG gene in the plants transformed with a vector in which the COG gene is inserted in the sense direction, or for inducing early flowering by inhibiting the expression of the COG gene in the plants by transforming them with a vector in which the COG gene is inserted in the antisense direction.

U.S. Patent Application Publication No. 2005/0060774 discloses isolated nucleic acids obtainable from the FRI (FRIGIDA) locus of plants which encode polypeptides capable of delaying the flowering time of a plant into which the nucleic acid is introduced.

COL (CONSTANS like protein) demonstrates high amino acid sequence homology with CO (CONSTANS), which plays a central role in the photoperiod response pathway by mediating between the circadian clock and the floral integrators (Suarez-Lopez et al., 2001). CO and COL form a family of 17 factors (Robson et al., 2001). The mRNA expression of CO, COL1 and COL2 follows a particular rhythm throughout the day, thus suggesting that it is controlled by the circadian clock (Ledger et al., 2001; Suarez-Lopez et al., 2001). In addition, the TAIR (The Arabidopsis Information Resource) microarray database revealed that COL5 was also expressed rhythmically, thereby leading to the expectation that the expressions of other COL mRNAs are also controlled by the circadian clock. CO, COL1, and COL2 are characterized genes in the family; however, the functions of the other members in this family are largely unknown.

One reference concerning COL is U.S. Patent Application Publication 2006/0059586, which discloses an isolated nucleic acid sequence encoding COL9 or COL10 and a transgenic plant transformed with the nucleic acid sequence. It is shown that flowering is delayed in a plant by introducing into the plant the isolated nucleic acid sequence encoding COL9 or COL10.

### SUMMARY OF THE INVENTION

In view of the above circumstances, exploring functions and their effects associated with the COL family has been of great interest among researches. In the present application, COL8 is chosen since it is one of the genes that were suggested to be controlled by the circadian clock.

The present invention provides an isolated nucleic acid including a nucleotide sequence encoding COL8, which controls hypocotyl length and flowering time in a plant. It is found that plants over-expressing COL8 under long day conditions show slight hypocotyl elongation and delayed flowering in comparison with the wild type. In addition, COL8 and FKF1, which is a circadian-clock related gene, are found to be localized at the same site in a plant cell, thereby indicating the existence of an interaction between the two.

The present invention further provides a method of producing a transformed plant with altered hypocotyl length and flowering time, comprising steps of: producing a vector including a nucleic acid having a nucleotide sequence encoding COL8, which controls hypocotyl length and flowering time; transforming agrobacterium with the vector for generating constructs; and transferring a selected construct into a plant. This method gives rise to hypocotyl elongation and delayed flowering in a plant, when the vector includes COL8 that is inserted in the sense direction and thus COL8 is over-expressed.

The present invention further provides a nucleic acid vector for obtaining a transformed plant with altered hypocotyls length and flowering time, comprising a nucleic acid having a nucleotide sequence encoding COL8.

The present invention further provides a transgenic plant transformed with a nucleic acid having a nucleotide sequence encoding COL8.

Those skilled in the art will appreciate these and other advantages and benefits of various embodiments of the invention upon reading the following detailed description of the preferred embodiments with reference to the below-listed drawings.

### BRIEF DESCRIPTION OF THE DRAWINGS

FIG. 1 shows the COL8 cDNA sequence (SEQ ID No. 1, the disclosure of which is hereby incorporated by reference) and amino acid sequences (SEQ ID Nos. 2-4, the disclosure of which are hereby incorporated by reference).

FIG. 2 shows the genealogical tree and protein structure of CO/COL Family.

FIG. 3A shows results of the analysis of COL8 expression using RT-PCR for the case of long day conditions.

FIG. 3B shows results of the analysis of COL8 expression using RT-PCR for the case of continuous illumination.

FIG. 3C shows results of the analysis of the expression of COL8 in individual organs, where R indicates roots; St, stems; RL, rosette leaves; CL, cauline leaves; F, flowers; Si, sheaths; and DS, dried seeds.

FIG. 4A shows selection of single-copy individuals by DNA gel blotting, where asterisks indicate intrinsic COL8 bands.

FIG. 4B shows selection of individuals over-expressing COL8 by RNA gel blotting.

FIG. 5 shows the hypocotyl length of individuals over-expressing COL8 under long day conditions, and a graph representing the mean values and SEs of 10 plants each.

FIG. 6A shows the growth status of plants grown for 5 weeks under long day conditions following low-temperature treatment, with the scale bar indicating 2 cm.

FIG. 6B shows comparison of the flowering time between WT (Col-O-G) and plants over-expressing COL8 under long day conditions. The graph on the left shows the number of days to flowering, and the graph on the right shows the number of rosette leaves at the time of flowering. Each graph shows the mean values and SEs of 10 plants each.

FIG. 7A shows selection of COL8 antisense individuals by RNA gel blotting.

FIG. 7B shows comparison of flowering time of WT (Col-O-G) and COL8 antisense individuals under long day conditions. The graph on the left shows the number of days to flowering, and the graph on the right shows the number of rosette leaves at the time of flowering. Each graph shows the mean values and SEs of 14-16 individuals each.

FIG. 8A shows photographs indicating localization of YFP-FKF1 and CFP-COL8 at the same site in onion epidermal cells.

FIG. 8B shows an enlarged view of the nuclei in FIG. 8A.

### DETAILED DESCRIPTION OF THE PREFERRED EMBODIMENTS

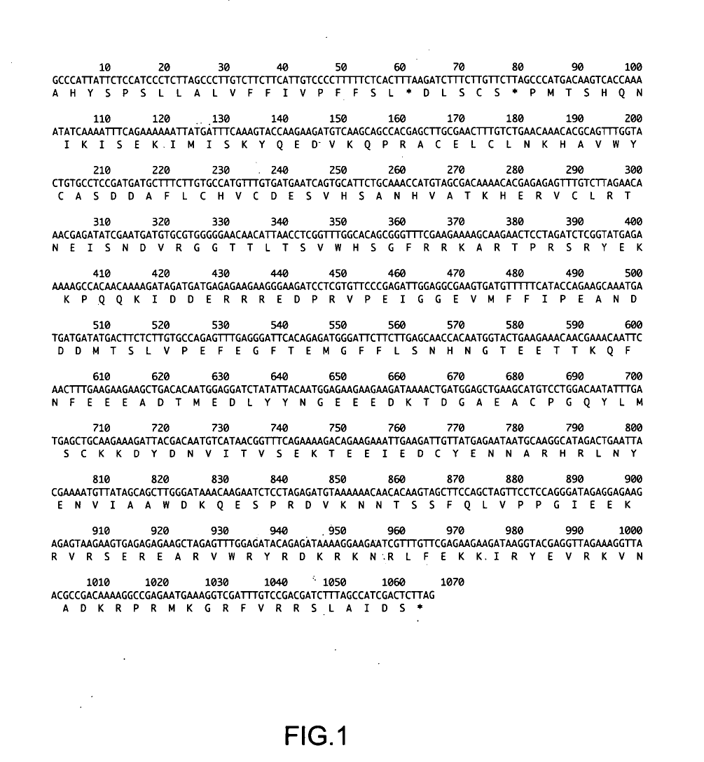

The present study was conducted for the purpose of analyzing the function of COL8. FIG. 1 shows the COL8 cDNA sequence and amino acid sequence. COL8 has the possibility of being controlled by the circadian clock; therefore, its expression rhythm was analyzed as follows.

Under light-dark conditions (16 hr light, 8 hr dark), a 24-hour cyclic expression rhythm was observed, which peaked in the vicinity of ZT20. In addition, although the same expression rhythm as that under the light-dark conditions was demonstrated for 0 to 24 hr during continuous illumination, the amplitude decreased considerably and the expression became nearly constant from 24 to 48 hr.

The expression of COL8 in each organ was analyzed. Although low expression levels were observed in the roots and dried seeds, roughly the same high expression levels were observed in the other organs. In addition, an analysis of the changes in the expression levels with age revealed that there were no remarkable difference in the expression levels between rosette leaves on day 17 and those on day 35.

Plants that over-expressed COL8 and those that over-expressed the antisense strand were prepared, and their phenotypes were observed. During the observation of the hypocotyl lengths of plants over-expressing COL8 under long day conditions (16 hr light, 8 hr dark), slight hypocotyl elongation was noted in comparison with the wild type (WT). In addition, an examination of flowering time revealed that flowering tended to be delayed for both lines of plants over-expressing COL8 under long day conditions. However, that there were no remarkable differences between the plants over-expressing the antisense strand and the WT suggested the existence of another complementary function.

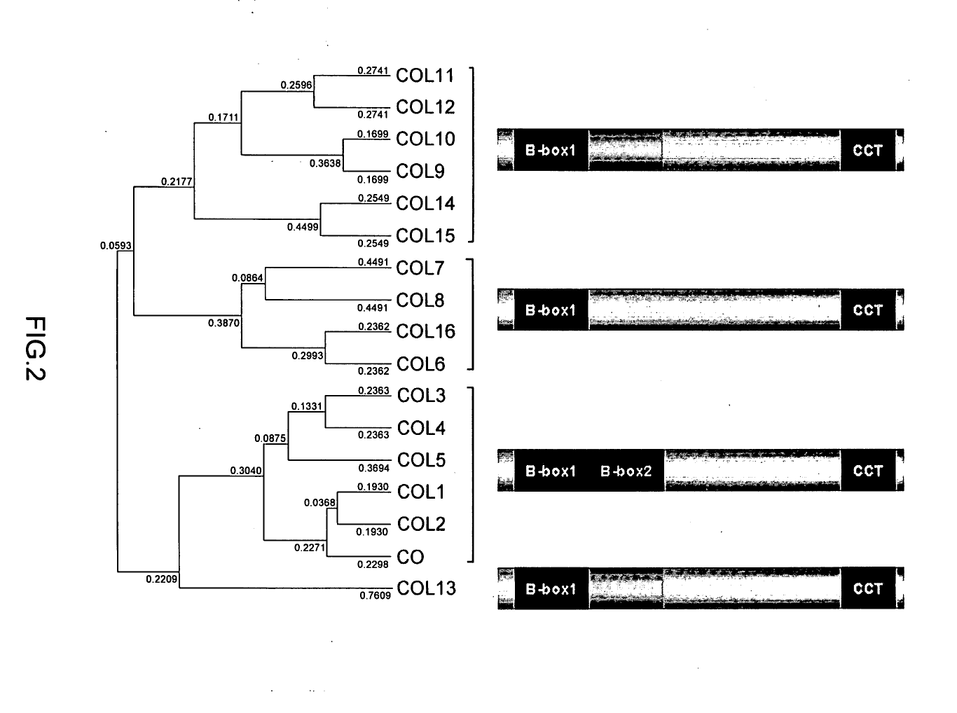

Using the yeast-two-hybrid method, we observed the interaction of several factors of the COL family, including CO, with the LKP family of proteins. FIG. 2 shows the genealogical tree and protein structure of CO/COL Family. The genealogical tree in FIG. 2 was prepared from the amino acid sequences of the CO/COL family using the UPGMA method (Fukamatsu et al. 2005).

COL8 was surmised to function at a position close to FKF 1 in particular among the LKP family of proteins because its expression is controlled by the circadian clock and light, and it is involved in flowering control. Therefore, when CFP-COL8 and YFP-FKF1 were simultaneously injected into onion epidermal cells, they were both found to be localized in the nucleus, their fluorescence coinciding.

In conclusion, the results suggest that the expression of COL8 may be controlled by the circadian clock or light, and be involved in hypocotyl elongation and flowering control.

Details in experiments and results are described in the following.

Cloning of COL8 cDNA

The entire length of COL8 cDNA was cloned by RT-PCR (Reverse Transcriptase-Polymerase Chain Reaction) and 5′-RACE (Rapid Amplification of 5′ Complementary DNA Ends). 5′-RACE was carried out using the 5′-RACE System Version 2.0 (Invitrogen). 1st strand cDNA was synthesized using GTGTCAGCTTCTTCTTCAAAGTTGAATTGT as the primer and RNA of Arabidopsis thaliana ZT20 (4 hr after lights were switched off) bred for 3 weeks under long day conditions (16 hr light, 8 hr dark) as the template. After treating with terminal deoxy-nucleotidyl transferase, PCR was carried out using GCACAAGAGAAGTCATATCATCATCATTTG and Abridge Anchor primers. Subsequently, the nested PCR was carried out using ATCTATCTTTTGTTGTGGCTTTTTCTCATA and AUAP primers, and the resulting fragment was subcloned using the pGEM-T Easy Vector System (Promega) and then sequenced.

2. Analysis of COL8 Expression by RT-PCR

Sampling

Plants grown for 9 days in MS (Murashige and Skoog, 1962) agar medium under long day conditions (16 hr light, 8 hr dark) following low-temperature treatment for 3 days after seeding were used for circadian rhythm experiments. Sampling was started at dawn on day 10 and carried out every 4 hr thereafter for 48 hr. Only the above-ground portions of the plants were sampled. All the plant organs used for expression analysis were sampled at 14:00 by taking into account the expected circadian rhythm of COL8. Roots and rosette leaves were sampled on day 17 following low-temperature treatment, whereas stems, rosette leaves, flowers and sheaths were sampled on day 35 following low-temperature treatment. In addition, rosette leaves were sampled twice on days 17 and 35 to examine differences in expression levels with age.

(2) Extraction of RNA from Arabidopsis Plants

The RNA used in this study was extracted with TRI REAGENT (Molecular Research Center, Inc.). When the plant weight exceeded 100 mg, the sampled organs were crushed with mortar and pestle, and when it was less than 100 mg, the sampled organs were crushed in an Eppen tube filled with glass beads.

(3) RT-PCR

RT-PCR was carried out in a single step using the Access Quick™ RT-PCR System (Promega). The primer sequences for the COL8 expression analysis were comprised of AGGATCCGTT TGATGAGCTG CAAGAAAGAT and AGGATCCCTA AGAGTCGATG GCTAAAGATC from the 5′ side to the 3′ side. In addition, CCGGAAAACA ATTGGAGGATGGT and GTCATTAGAA AGAAAGAGATAAC were used from the 5′ side to the 3′ side to amplify UBQ10 used as control. The reaction system was comprised of 7.6 μl of DEPC-H₂O, 0.4 μg of total RNA, 1 μl of forward primer (10 μM), 1 μl of reverse primer (10 μM), 10 μl of 2×AQ buffer and 0.4 μl of AMV reverse transcriptase. The reaction proceeded as follows: reverse transcription for 45 minutes at 48° C. and heat denaturation for 2 minutes at 95° C. to deactivate the reverse transcriptase. RT-PCR was then carried out for 30 cycles including denaturation for 30 seconds at 95° C., annealing for 60 seconds at 57° C. and elongation for 90 seconds at 72° C. RT-PCR for UBQ10 was carried out for 20 cycles at an annealing temperature of 47° C.

(4) Electrophoresis

COL8 and UBQ10 were electrophoresed on 1.2 to 1.5% agarose gel (w/v). Following electrophoresis, the samples were stained with EtBr solution and the bands were detected by UV irradiation.

(5) Quantification of PCR-Amplified Fragments

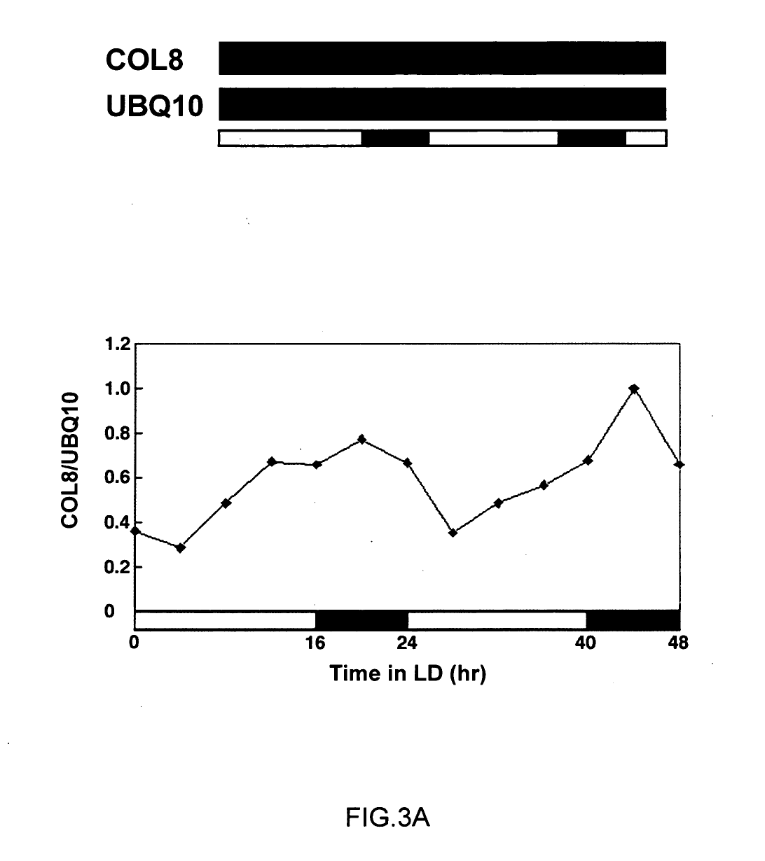

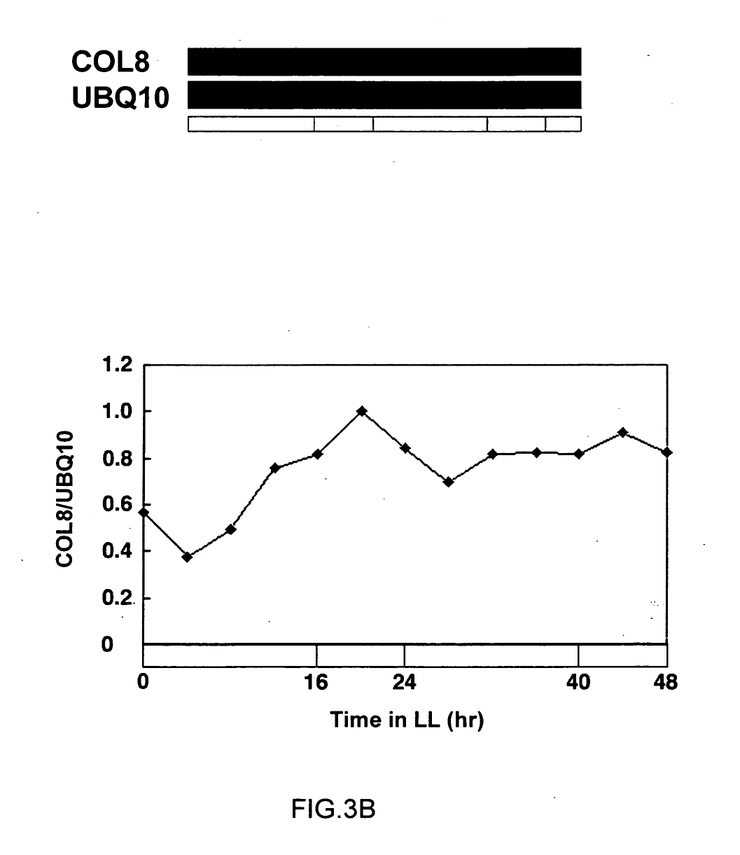

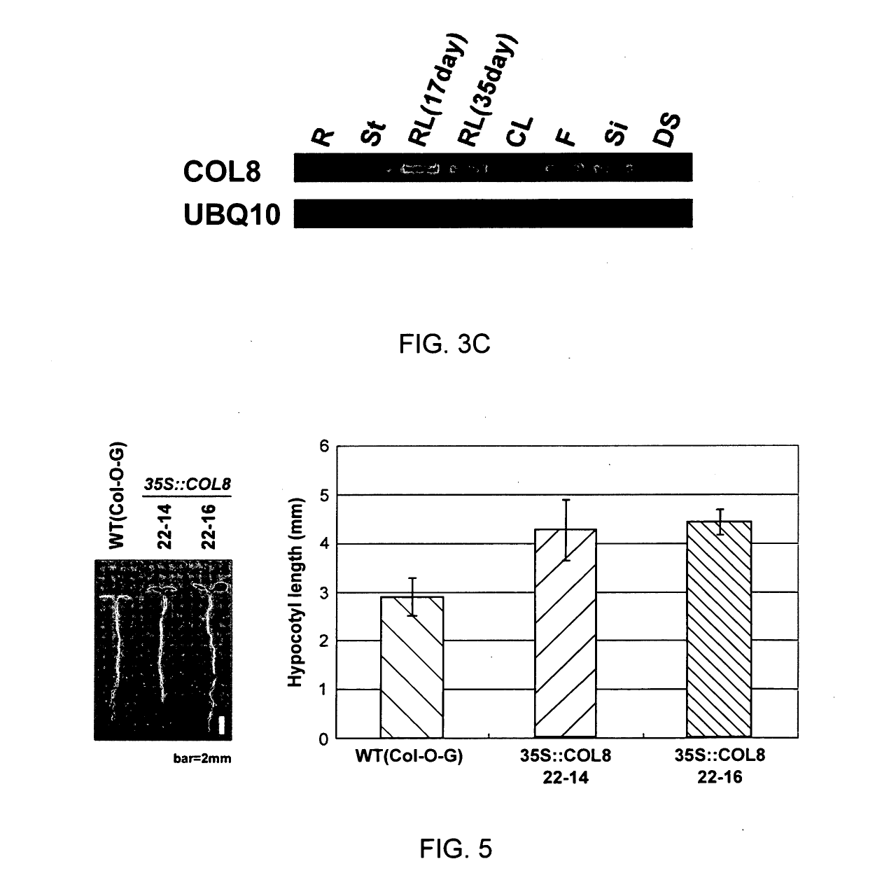

The CS Analyzer (ATTO) was used to quantify the amplified fragments of COL8 and UBQ10. The calculated values of the COL8 amplified fragments were divided by the values of the UBQ10 amplified fragments and plotted in FIGS. 3A and 3B.

As shown in FIG. 3A, under 16-hr light/8-hr dark conditions, a 24-hour cyclic expression rhythm was observed, peaking in the vicinity of ZT20. On the other hand, when the expression rhythm was examined under continuous illumination, although COL8 demonstrated the same expression rhythm as that of the light-dark conditions for the first 24 hr, its amplitude was decreased considerably and the expression became nearly constant thereafter, as can be seen in FIG. 3B.

Since no well-defined expression rhythm was observed under continuous illumination, it was hypothesized that the expression rhythm of COL8 is dependent on the light-dark cycle. In order to verify this hypothesis, it will be necessary to analyze the expression under conditions of continuous darkness and short day conditions.

Moreover, the expression levels of RNA extracted from the roots, stems, rosette leaves, cauline leaves, flowers, sheaths and dried seeds were analyzed by RT-PCR. Although the expression levels in the roots and dried seeds were extremely low, the expression levels were roughly the same in the other organs, as shown in FIG. 3C. In addition, since no remarkable differences in the expression levels were observed between rosette leaves on days 17 and 35, it was surmised that there were no differences in the expression levels with age following germination.

3. Analysis of Plants Over-expressing COL8

Although the analysis of individuals containing COL8 T-DNA inserts was attempted, there were no individuals in which the T-DNA was inserted at a suitable position Thus, the analysis of COL8 through the Loss-of-Function approach was aborted. Instead, plants over-expressing COL8 were produced and analyzed for the purpose of pursuing the analysis through the Gain-of-Function approach.

Production of Constructs for Producing Plants Over-expressing COL8

The pBE2113-N vector (Mitsuhara et al., 1996) was used to produce plants over-expressing COL8. COL8 cDNA was amplified from mRNA by RT-PCR, using AGGATCCGTATGATTTCAAAGTACCAAGAA and AGGATCCCTAAGAGTCG ATGGCTAAAGATC as the primers, and cloned to pGEM-T Easy Vector. This was followed by confirmation of the absence of errors by sequencing.

After treating with BamHI, the COL8 cDNA fragment that had been purified by cleaving from the cloning vector with BamHI was ligated to the alkaline-phosphatase-treated pBE2113-N vector, and then used to transform Escherichia coli. The resulting colonies were selected arbitrarily and subjected to plasmid extraction with PI50α. The plasmids were treated with a suitable restrictase and subjected to PCR to confirm that the insert had been inserted in the sense direction under the control of the CaMV 35S promoter.

The construct that was determined to be correct using the Agrobacterium method was transferred into the plants (Columbia ecotype) using the Floral dip method (Clough and Bent, 1998).

2) Selection of Plants Over-expressing COL8

Probe Production

The 262 bp to 568 bp of COL8 cDNA sequence was used as the probe in RNA gel blotting. XhoI-treated pGADT7/COL8 (262 bp to 568 bp) was purified by phenol-chloroform treatment, and a single-strand plasmid was recovered by ethanol precipitation. The resulting DNA was dissolved in DEPC-H₂O to a suitable concentration. This was then labeled with DIG using the AmpliScribe™ T7 High Yield Transcription Kit (Epicentre).

(2) Electrophoresis

Agarose gel (5% 20×MOPS (0.4 M MOPS, 0.1 M sodium acetate, 0.02 M EDTA), 1.0% agarose, 5% formaldehyde) was poured into a gel plate and a sample comb was placed on it. After solidifying, the sample was immersed for about 30 minutes in buffer (5% 20×MOPS). RNA sample buffer (50% glycerin, 0.1 mg/ml BPB, 0.1 mg/ml xylene cyanol, 1 mM EDTA) was added to 5 μl of sample containing 1 μg of RNA. After stirring well, the mixture was spun down and allowed to stand for 10 minutes at 65° C. and then for 5 minutes on ice. This was then applied to the agarose gel and electrophoresed at 100 V and 70 mA. Following electrophoresis, the gel was stained with EtBr to obtain an RNA electrophoresis pattern by UV irradiation. This gel was washed for 60 minutes with 20×SSC (3 M NaCl, 0.3 M sodium citrate-2H₂O, pH 7.0) and blotted.

(3) Membrane Transfer

A bridge made with an acrylic plate was placed in a device containing 20×SSC. Two pieces of 3 MM filter paper of a length that allowed adequate immersion in the 20×SSC, gel, membrane and another two pieces of 3 MM filter paper were layered on this acrylic plate in that order. Thereafter, pieces of tissue paper and Kimwipe cut to the same size as the filter paper were layered on top and allowed to stand overnight with an acrylic plate and a weight on it. The membrane was stabilized by UV-crosslinking the following day.

(4) Hybridization

DIG Easy Hyb (Roche) was used as the hybridization buffer. The membrane was placed in a HybriPack, and this was followed by the addition of DIG Easy Hyb (15 ml/100 cm²) and pre-hybridization for 30 minutes at 68° C. After initially discarding the buffer, DIG Easy Hyb (3.5 ml/100 cm², 250 ng/ml RNA probe) was added, followed by hybridizing overnight at 68° C. The membrane was removed the following day, washed twice at room temperature for 15 minutes with low-stringency buffer (0.1% SDS, 2×SSC), and then washed twice at 68° C. for 15 minutes with high-stringency buffer (0.1% SDS, 0.1×SSC). After washing for 2 minutes with washing buffer (0.1 M maleic acid, 0.15 M NaCl, 0.3% (v/v) Tween 20, pH 7.5), blocking was carried out for 1 hour with blocking buffer (Roche).

Next, the membrane was agitated for 30 minutes with 150 ml of DIG antibody solution (15 μl of DIG antibody, 150 ml of blocking buffer). Subsequently, the membrane was washed twice for 15 minutes with washing buffer and then agitated for 5 minutes with detection buffer (0.1 M Tris-HCl, 0.1 M NaCl, pH 9.5). The membrane was then transferred to a HybriPack and a suitable amount of CDP-Star was spread onto its surface, and after allowing to stand undisturbed for 5 minutes, chemiluminescence was detected with Cool Sever (Atto).

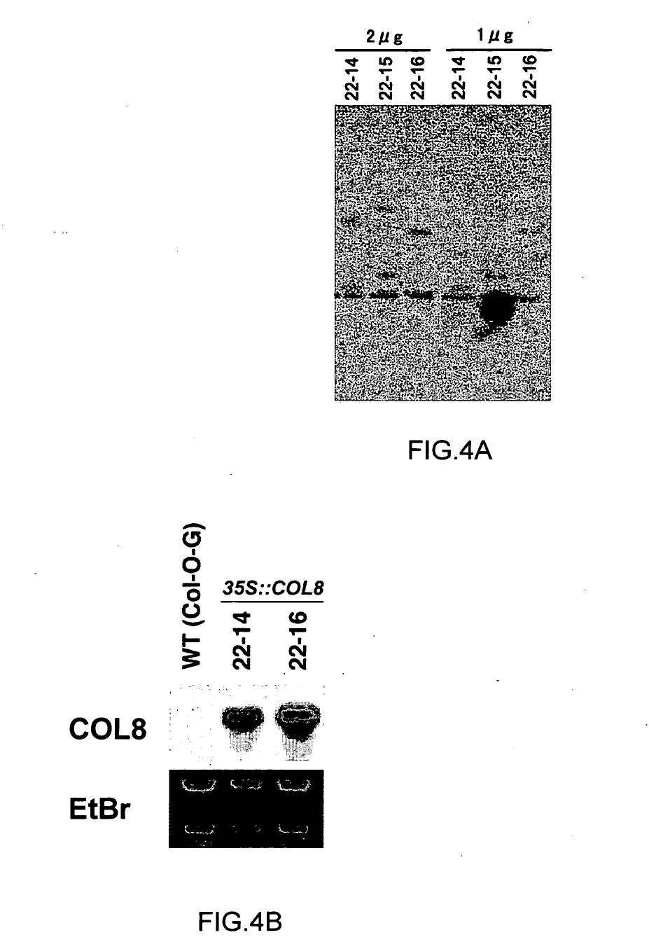

As shown in FIGS. 4A and 4B, the individual COL8 over-expressing plants used for analysis had two homogeneous lines containing a single location where T-DNA was inserted. FIG. 4A shows the selection of single-copy individuals by DNA gel blotting. Two μg of genomic DNA was used as template for the three left lanes, whereas 1 μg of genomic DNA was used as template for the three right lanes. Asterisks indicate intrinsic COL8 bands. FIG. 4B shows the selection of individuals over-expressing COL8 by RNA gel blotting. Total RNA was extracted from individuals grown for 2 weeks on MS agar medium following low-temperature treatment, followed by RNA gel blotting using 2 μg as template.

3) Selection of Single Locus Individuals by DNA Gel Blotting

Extraction of Genomic DNA and Restrictase Treatment

The plant body was pulverized in liquid nitrogen and dissolved in 3 ml of 3×CTAB solution that was warmed to 60° C. (100 mM Tris-HCl, 1.4 M NaCl, 20 mM EDTA (pH 8.0), 3% CTAB). After incubating for 30 minutes at 60° C., 3 ml of chloroform was added, followed by mixing by inverting about 20 times. After centrifuging for 5 minutes at 5000 rpm, the supernatant was transferred to an Eppen tube. 2-Propanol was added to the supernatant in an amount equal to 70% of the solution and allowed to stand undisturbed for 15 minutes at room temperature. After centrifuging again for 5 minutes at 5000 rpm, 70% ethanol was added, and this was followed by rinsing, air-drying and dissolving in 50 μl of TE. This was used for restrictase treatment following RNase treatment.

A single HindIII site is present in the T-DNA of the pBE2113-N vector, and as a result of cleavage with HindIII, the number of copies of T-DNA can be determined. The reaction system consisted of 2 μl of 10×M buffer, 2 μl of HindIII, 1.5 μl of 100 mM spermidine and 2 μg of template DNA, and it was incubated overnight at 37° C. The reaction was then carried out for 5 minutes at 70° C. to deactivate the HindIII.

(2) Electrophoresis

Agarose gel (2% 50×TAE, 0.8% agarose) was poured into a gel plate and a sample comb was placed on it. Two μg of genomic DNA treated with restrictase was applied to the agarose gel and electrophoresis was conducted at 100 V and 70 mA. The gel was then stained with EtBr and the genomic DNA was confirmed to have been cleaved by UV irradiation. After washing the gel with distilled water, it was treated for 15 minutes with 500 ml of hydrolysis solution (0.25 N HCl). The gel was then denatured for 30 minutes with 500 ml of denaturing solution (0.5 N NaOH, 1.5 M NaCl) and neutralized for 30 minutes with neutralizing solution (0.5 M Tris-HCl, 1.5 M NaCl), followed by blotting.

(3) Blotting

A bridge made with an acrylic plate was placed in a device containing phosphate buffer (25 mM NaPO₄, pH 6.6). Two pieces of 3 MM filter paper of a length that allowed adequate immersion in the phosphate buffer, gel, membrane and another two pieces of 3 MM filter paper were layered on this acrylic plate in that order, after which pieces of tissue paper and Kimwipe cut to the same size as the filter paper were layered on top and allowed to stand overnight with an acrylic plate and a weight on it. The membrane was stabilized by UV-crosslinking the following day.

(4) Hybridization

Hybridization and probe production were carried out using the AlkPhos Direct Labeling and Detection System (Amersham). Following hybridization, fluorescence was detected using Cool Sever (Atto).

4) Selection of Homogeneous Individuals Based on Separation Ratio

Southern hybridization was carried out to investigate the separation ratio by seeding 100 T3 seeds, each in MS-Km medium, for those lines that demonstrated a single copy of T-DNA. The lines in which the ratio of Km-resistant individuals to non-Km-resistant individuals was 1:0 were treated as homogeneous lines and used for the analysis of Transgenic 3 (T3) plants.

5) Observation of T3 Plants

Measurement of Hypocotyl Length

Seeds were seeded in sucrose-free MS agar medium and subjected to low-temperature treatment for 4 days in the dark at 4° C. Following the low-temperature treatment, the seeds were irradiated for 2 hr with white light at 30 μmol m⁻²s⁻¹ and 22° C. to induce germination, and then subjected to dark treatment for 24 hr. The seeds were then grown for 5 days under long day conditions (16 hr light, 8 hr dark, light intensity: 75 μmol m⁻² s⁻¹). The plants that grew were photographed with a digital camera and hypocotyl lengths were measured with NIH Image.

FIG. 5 shows the hypocotyl length of individuals over-expressing COL8 under long day conditions. The graph shows hypocotyl lengths after seeding onto MS-sucrose agar medium, subjecting to low-temperature treatment for 4 days, and growing for 5 days under 16-hr light/8-hr dark conditions. The graphs show the mean values and SEs of 10 plants each. Changes in hypocotyl length were observed under white light, red light and blue light among functionally deficient plants of the LKP family and plants over-expressing COL8. When the hypocotyl lengths of plants over-expressing COL8 were analyzed under long day conditions (16 hr light, 8 hr dark), a slight hypocotyl elongation was observed in comparison with the WT, as shown in FIG. 5. Hypocotyl elongation is considered to be controlled by signals from light receptors. Thus, analyses under various light conditions are needed to elucidate the functional pathways of COL8.

(2) Measurement of Flowering Time

Vermiculite mixed with 1000-fold diluted Hyponex was packed into pots and each pot was seeded with a single seed. Following low-temperature treatment in the dark at 4° C. for 3 days, the seeds were grown at 22° C. under long day conditions (16 hr light, 8 hr dark, light intensity: 75 μmol m⁻²s⁻¹). A suitable volume of water containing Hyponex diluted to 1/1000 by volume was added once every 4 days. Sprouting was assumed to have occurred at the point the initial stem reached a length of 1 cm, and the number of rosette leaves at that time and the number of days after transferring to long day conditions were recorded. Flowering was assumed to have occurred at the time the first flower bloomed, and the number of rosette leaves at that time, the number of cauline leaves that grew from the initially sprouted stem, and the number of days after transferring to long day conditions were recorded. Ten individuals were observed for each line, and data are shown as mean values and standard errors (SE).

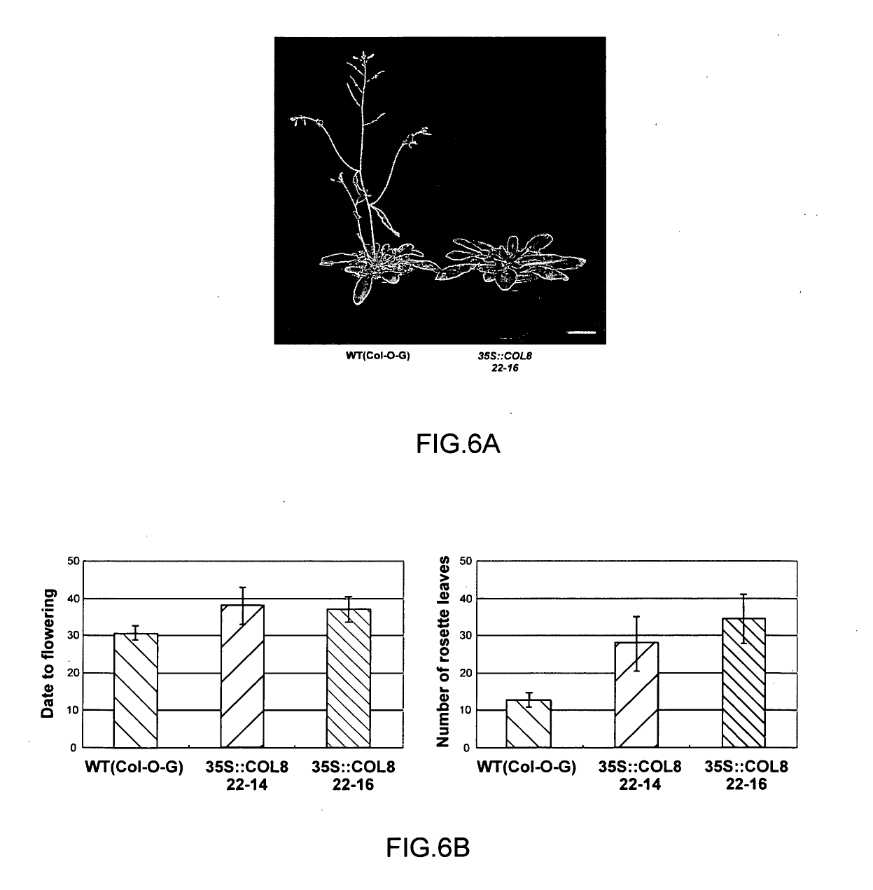

FIG. 6A shows the growth status of plants grown for 5 weeks under long day conditions following low-temperature treatment. The scale bar indicates 2 cm. FIG. 6B shows comparison of the flowering time between WT (Col-O-G) and plants over-expressing COL8 under long day conditions. The graph on the left shows the number of days to flowering, and the graph on the right shows the number of rosette leaves at the time of flowering. Each graph shows the mean values and SEs of 10 plants each. Examination of the flowering time indicated that flowering was delayed under long day conditions (16 hr light, 8 hr dark), as shown in FIGS. 6A and 6B.

Table 1 shows the number of rosette leaves and the number of cauline leaves at the time of flowering, and the number of days to flowering after low-temperature treatment for individuals over-expressing COL8. The values represent the mean values and SEs of 10 individuals each.

TABLE 1

| | No. of | | No. of | | | | |
|---|---|---|---|---|---|---|---|
| | Rossette | | Cauline | | Date to | | |
| Plant | Leaves | SE | Leaves | SE | Flowering | SE | n |
| WT (Col-O-G) | 12.8 | 1.81 | 3 | 0 | 30.6 | 1.78 | 10 |
| 35S::COL8 | 27.8 | 7.21 | 3.9 | 1.1 | 38 | 4.71 | 10 |
| 22-14 | | | | | | | |
| 35S::COL8 | 34.4 | 6.44 | 3.8 | 0.79 | 37 | 3.43 | 10 |
| 22-16 | | | | | | | |

From this Table, it can be seen that the number of rosette leaves was increased whereas that of cauline leaves was unchanged compared with the WT. Therefore, there is a mild delay in flowering compared with plants over-expressing LKP1 or LKP2 and FKF1 plants.

4. Analysis of COL8 Antisense Plants

Production of Constructs for Producing COL8 Antisense Plants

Plants over-expressing COL8 were produced using the pBE2113-N vector. After treating with BamHI, COL8 cDNA fragments that were purified by cleaving from a cloning vector with BamHI were ligated to the alkaline-phosphatase-treated pBE2113-N vector and then used to transform E. coli. The resulting colonies were arbitrarily selected and the plasmids were extracted with PI50α. These plasmids were then treated with a suitable restrictase and subjected to PCR to confirm that the inserts were inserted in the reverse direction under the control of the CaMV 35S promoter. The correct construct was then transferred into the plant (Colombia ecotype) using the Agrobacterium method.

2) Observation of Antisense Individuals

Breeding of T1 and T2 Generation Plants

The seeded seeds were allowed to stand for 3 days in the dark at 4° C., and then grown under long day conditions (16 hr light, 8 hr dark) at 22° C. and a light intensity of approximately 75 μmol m⁻²s⁻¹ (measuring range: 400-700 nm, measuring instrument: Basic Quantum Meter). Transgenic 1(T1) and Transgenic 2 (T2) seeds were selected in MS agar medium containing kanamycin (25 mg/ml) and the plants for which 2 hr had elapsed following low-temperature treatment were grown by replanting in pots containing vermiculite.

(2) Selection of COL8 Antisense Individuals by RNA Gel Blotting

The entire length of COL8 was used to select individuals over-expressing the antisense strand. pCR4/COL8 was treated with PstI, purified with phenol-chloroform treatment, and subjected to ethanol precipitation to recover the single strand plasmid. The resulting DNA was dissolved to a suitable concentration in DEPC-H₂O. This was then labeled with DIG using the AmpliScribe™ T7 High Yield Transcription Kit (Epicentre). The subsequent process included performing the same procedure as that used for individuals over-expressing COL8, followed by the selection of individuals that over-expressed the antisense strand.

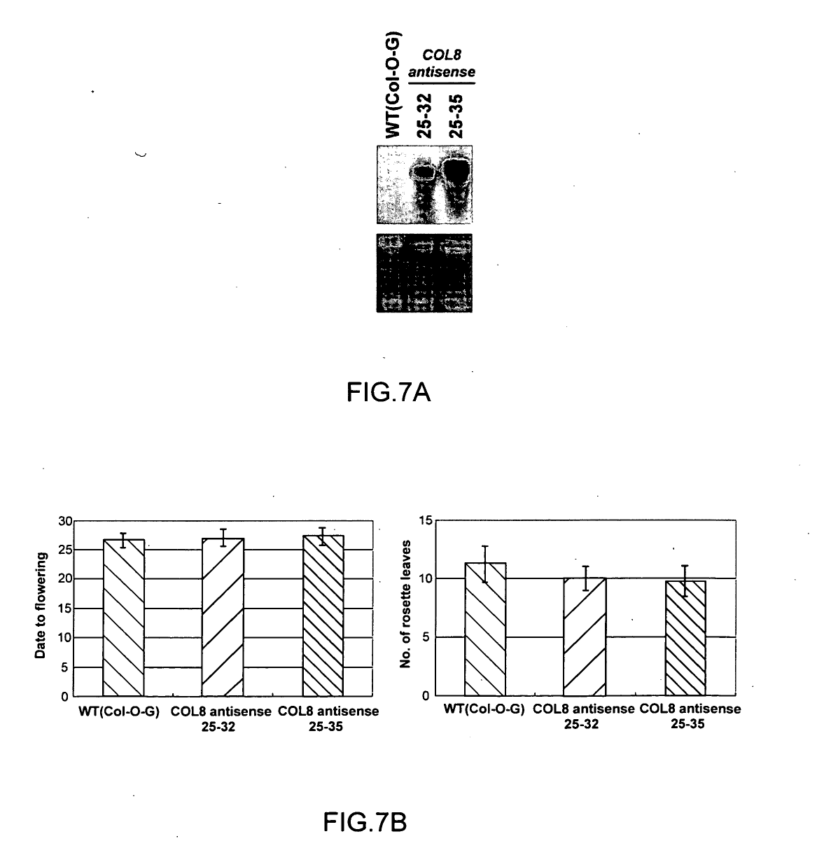

T2 generation plants were seeded for two lines of plants that over-expressed the antisense strand in the T1 generation as shown in FIG. 7A.

(3) Observation of Individuals Overxpressing Antisense Strand

Individuals over-expressing the antisense strand were observed by comparison with the WT (Col-O-G). Flowering time in the T2 generation was measured using two lines that over-expressed the antisense strand in the T1 generation. The same method as that for analyzing individuals over-expressing COL8 was used to measure flowering time.

FIG. 7A shows selection of COL8 antisense individuals by RNA gel blotting. Total RNA was extracted from individuals grown for 30 days following low-temperature treatment and subjected to RNA gel blotting using 1 μg as template. FIG. 7B shows comparison of flowering time of WT (Col-O-G) and COL8 antisense individuals under long day conditions. The graph on the left indicates the number of days to flowering, and the graph on the right indicates the number of rosette leaves at the time of flowering. Each graph shows the mean values and SEs of 14-16 individuals each.

As shown in FIG. 7B, no significant differences were observed in comparison with the WT. It was surmised that the phenotype did not appear because the COL family has factors with overlapping functions, whereas the phenotype appeared as a result of crossing with other individuals in which COL expression was suppressed. In addition, since it is also possible that COL8 expression was not suppressed even though the antisense strand is over-expressed, it will be necessary to analyze COL8 expression in antisense individuals.

5. Cell Localization Analysis Using Fluorescent Protein

As described in the section 4 above, the experimental result suggested the interaction between COL8 and FKF1. In order to confirm that there is indeed an interaction between the two, the following experiment was performed using YFP (yellow fluorescent protein) and CFP (cyan fluorescent protein), which serve as reference markers for FKF1 and COL8 respectively. These markers enable visual observation of localization. It should be noted here that this experiment was conducted based on the assumption that an interaction exists between these two proteins if they are localized at the same cite in a cell.

Production of YFP/CFP Fusion Constructs

pAVA594. was used as the vector for fusion protein expression with 35S::YSP, whereas pAVA554 was used as the vector for fusion protein expression with 35S::CFP (von Arnim et al., 1988). Each vector encodes a BglII domain downstream of 35S::YFP and 35S::CFP. Each vector was subjected to Klenow treatment following restrictase treatment with BglII. Klenow treatment was carried out in a reaction system consisting of 1.0 μl of 10×buffer, 1.0 μl of dNTP, 0.5 μl of Klenow fragment and 200 ng of template brought to a total volume of 10 μl by the addition of sterile water. The reaction conditions included reaction for 30 minutes at 16° C., followed by deactivation for 10 minutes at 72° C. Subsequently, alkaline phosphatase treatment was performed following ethanol precipitation to produce the vectors. BamHI sites were added to both ends of the COL8 cDNA fragments, and this was followed by Klenow treatment using the same method as that for the vectors. Thereafter, the fragments were used in a ligation reaction. In addition, an NcoI site originating in the pGEM vector was added to the 5′ side of the FKF1 cDNA fragment, whereas an SpeI site was added to the 3′ side, and these were similarly subjected to Klenow treatment, followed by use in a ligation reaction. Following the ligation reaction, the COL8 cDNA fragments were used to transform E. coli, and this was followed by plasmid extraction using PI50α (Kurabo Industries). Then, treatment with a suitable restrictase and sequence analysis were conducted to confirm that the insert was correctly inserted. The resulting plasmids were subjected to ethanol precipitation and used for particle bombardment after adjusting to a final concentration of 2 μg/μl.

(2) Particle Bombardment

First, gold particles were added to 50% glycerol and subjected to vortex mixing until the gold particles were uniformly mixed. Once the gold particles were uniformly mixed, 6.25 μl was transferred to an Eppen tube, and this was followed by the addition of 1 μl (2 μg) each of the CFP/YFP fusion plasmids and the pBI221/RFP plasmids, 6.25 μl of 2.5 M CaCl₂ and 20 μl of 0.1 M spermidine, and vortexing for 2 to 3 minutes. After allowing to stand undisturbed for 1 minute, the mixture was centrifuged for 2 minutes at 15000 rpm. After removing the supernatant, 140 μl of 70% ethanol was added to wash the inside of the Eppen tube without breaking the pellet. After a similar wash with 100% ethanol, 10 μl of 100% ethanol was added to break up the pellet, and this was followed by spotting onto a macro carrier while taking care to ensure that the gold particles were uniformly mixed. After the gold particles were dried completely, they were injected into onion epidermal cells at 1550 PSI using PDS-1000/He (Bio-Rad). The injected onion was stored in a Petri dish, protected from the light with aluminum foil and incubated overnight at 22° C.

(3) Microscopic Observation of Fluorescence

The epidermal layer was peeled from the onion following incubation, placed on a glass slide and observed with a BX51 microscope (Olympus). The resulting images were analyzed with MetaMorph (Universal Imaging Corporation™). The experiment was repeated three to four times for a single sample to confirm reproducibility (Fukumatsu et al., 2005).

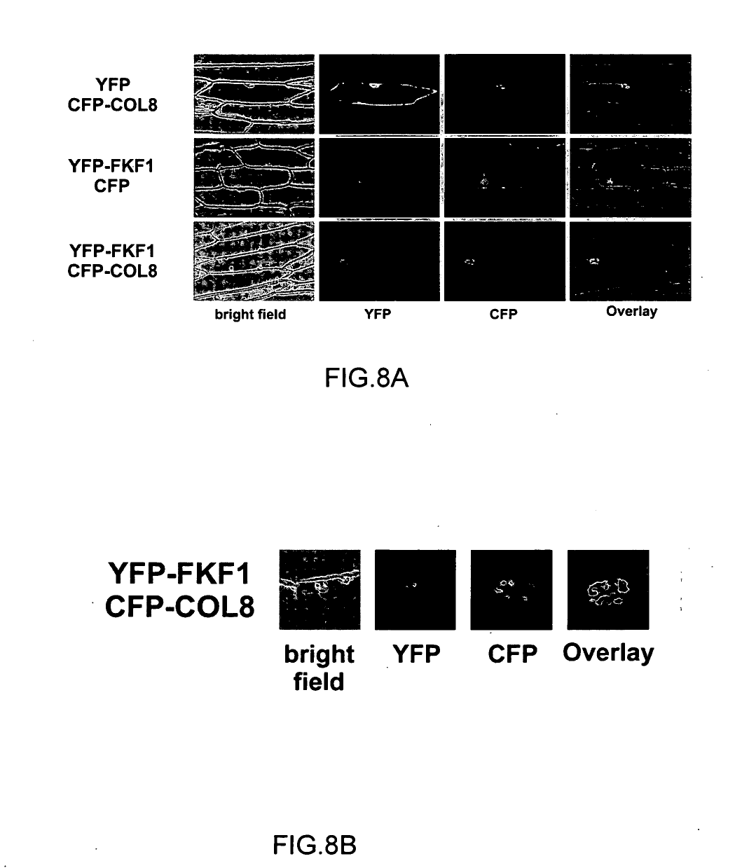

FIG. 8A shows photographs indicating the localization of YFP-FKF1 and CFP-COL8 in onion epidermal cells. FIG. 8B shows an enlarged view of the nuclei in FIG. 8A. As shown in these photographs, when YFP-FKF1 and CFP-COL8 were simultaneously injected into onion epidermal cells, both were found to be localized at the same site in the nucleus, thereby indicating that FKF1 and COL8 interact with each other.

On the basis of these findings, it was surmised that the expression of COL8 may be controlled by the circadian clock or light-dark conditions, and is involved in the control of hypocotyl elongation and flowering time. We have found that the FKF/LKP/ZTL family proteins interact with the PRR/TOC1 family proteins, and are involved in the control of circadian rhythm, hypocotyl elongation and flowering time (Kiyosue and Wada, 2000; Schultz et al., 2001; Yasuhara et al., 2004).

In addition, since not only COL8 but also CO and other COL proteins have been shown to interact with the FKF/LKP/ZTL family proteins (Fukumatsu et al., 2005), it is possible that the FKF/LKP/ZTL family proteins are involved in the control of hypocotyl elongation and flowering time through the CO/COL family proteins.

It is to be understood that the above-described embodiments are illustrative of only a few of the many possible specific embodiments which can represent applications of the principles of the invention. Numerous and varied other arrangements can be readily devised by those skilled in the art without departing from the spirit and scope of the invention.

### REFERENCES

The references listed below are incorporated herein by reference to the extent that they supplement, explain, provide background for, or teach methodology, techniques, and/or compositions employed therein.

U.S. Pat. No.6,265,637

U.S. Pat. No.6,689,940

U.S. Pat. No. 6,727,407

U.S. Pat. No. 6,586,252

U.S. Patent Application Publication No. 2005/0066393

U.S. Patent Application Publication No. 2005/0183166

U.S. Patent Application Publication No. 2005/0060774

U.S. Patent Application Publication No. 2006/0059586

Blazquez, M. A., “Flower development pathways,” J. Cell Sci. 113, 2000, pp. 3547-3548.

Clough S. J., Bent A. F., “Floral dip: a simplified method for Agrobacterium-mediated transformation of Arabidopsis thaliana;” Plant J. 16(6), 1998, pp. 735-743.

Fukamatsu Y., Mitsui S., Yasuhara M., Tokioka Y., Ihara N., Fujita S., Kiyosue T, “Identification of LOV KELCH PROTEIN2 (LKP2)-Interacting Factors that Can Recruit LKP2 to Nuclear Bodies” Plant Cell Physiol. Jun. 4, 2005 [Epub ahead of print].

Kiyosue T., Wada M., “LKP1 (LOV kelch protein 1): a factor involved in the regulation of flowering time in Arabidopsis,” Plant J. 23(6), 2000, pp. 807-815.

Ledger S., Strayer C., Ashton F., Kay S. A., Putterill J., “Analysis of the function of two circadian-regulated CONSTANS-LIKE genes,” Plant J. 26(1), 2001, pp. 15-22.

Mitsuhara I., Ugaki M., Hirochika H., Ohshima M., Murakami T., Gotoh Y, Katayose Y, Nakamura S., Honkura R., Nishimiya S., Ueno K., Mochizuki A., Tanimoto H., Tsugawa H., Otsuki Y, Ohashi Y., “Efficient promoter cassettes for enhanced expression of foreign genes in dicotyledonous and monocotyledonous plants,” Plant Cell Physiol. 37(1), 1996, pp. 49-59.

Murashige T., Skoog F., “A revised medium for rapid growth and bioassays with tobacco cultures,” Physiol. Plant. 15, 1962, pp. 473-97.

Robson F., Costa M. M., Hepworth S. R., Vizir I., Pineiro M., Reeves P. H., Putterill J., Coupland G., “Functional importance of conserved domains in the flowering-time gene CONSTANS demonstrated by analysis of mutant alleles and transgenic plants,” Plant J. 28(6), 2001, pp. 619-31.

Schultz T. F., Kiyosue T., Yanovsky M., Wada M., Kay S. A., “A role for LKP2 in the circadian clock of Arabidopsis,” Plant Cell. 13(12), 2001, pp. 2659-70.

Suarez-Lopez P., Wheatley K., Robson F., Onouchi H., Valverde F., Coupland G., “CONSTANS mediates between the circadian clock and the control of flowering in Arabidopsis,” Nature 410(6832), 2001, pp. 1116-20.

von Arnim A. G, Deng X. W., Stacey M. G., “Cloning vectors for the expression of green fluorescent protein fusion proteins in transgenic plants,” Gene 221, 1998, pp. 35-43.

Yasuhara M., Mitsui S., Hirano H., Takanabe R., Tokioka Y., Ihara N., Komatsu A., Seki M., Shinozaki K., Kiyosue T., “Identification of ASK and clock-associated proteins as molecular partners of LKP2 (LOV kelch protein 2) in Arabidopsis,” J. Exp. Bot. 55(405), 2004, pp. 2015-27.

What is claimed is:

1. An isolated nucleic acid comprising:
a nucleotide sequence encoding COL8 of SEQ ID NO:1, which controls hypocotyl length and flowering time in a plant.

2. The isolated nucleic acid according to claim 1, wherein over-expression of COL8 results in hypocotyl elongation and delayed flowering in a plant.

3. The isolated nucleic acid according to claim 2, wherein COL8 interacts with FKF1, which is a circadian-clock related gene, in a plant cell.

4. The isolated nucleic acid according to claim 1, wherein the nucleic acid is isolated from Arabidopsis thaliana.

5. A method of producing a transformed plant with altered hypocotyl length and flowering time, comprising steps of:
producing a vector having a construct including a nucleotide sequence encoding COL8 as claimed in claim 1, which controls hypocotyl length and flowering time;
transforming agrobacterium with said vector; and
transferring said construct into a plant by agrobacterium.

6. The method of producing a transformed plant with altered hypocotyl length and flowering time according to claim 5, wherein the vector includes COL8 that is inserted in sense direction and thus COL8 is over-expressed to obtain hypocotyl elongation and delayed flowering.

7. A vector for obtaining a transformed plant with altered hypocotyl length and flowering time, comprising a nucleotide sequence encoding COL8 as claimed in claim 1, which controls hypocotyl length and flowering time.

8. A transgenic plant transformed with a nucleic acid having a nucleotide sequence encoding COL8 as claimed in claim 1, which controls hypocotyl length and flowering time.

9. A transgenic plant according to claim 8, wherein over-expression of COL8 results in hypocotyl elongation and delayed flowering in a plant.

10. A protein produced by expression of a nucleic acid having a nucleotide sequence encoding COL8, which elongates hypocotyl length and delays flowering time.

11. A composition comprising:
a protein produced by expression of a nucleic acid having a nucleotide sequence encoding COL8, which controls hypocotyl elongation and flowering time; and
a physically acceptable carrier.


SEQUENCE LISTING (4 sequences)

SEQ ID NO: 1 - DNA, 1065 bp, Arabidopsis thaliana
  CDS (1)..(60)
  CDS (64)..(78)
  CDS (82)..(1062)
gcccattattctccatccctcttagcccttgtcttcttcattgtcccctttttctcactttaagatctttcttgttcttagcccatgacaagtcaccaaaatatcaaaatttcagaaaaaattatgatttcaaagtaccaagaagatgtcaagcagccacgagcttgcgaactttgtctgaacaaacacgcagtttggtactgtgcctccgatgatgctttcttgtgccatgtttgtgatgaatcagtgcattctgcaaaccatgtagcgacaaaacacgagagagtttgtcttagaacaaacgagatatcgaatgatgtgcgtgggggaacaacattaacctcggtttggcacagcgggtttcgaagaaaagcaagaactcctagatctcggtatgagaaaaagccacaacaaaagatagatgatgagagaagaagggaagatcctcgtgttcccgagattggaggcgaagtgatgtttttcataccagaagcaaatgatgatgatatgacttctcttgtgccagagtttgagggattcacagagatgggattcttcttgagcaaccacaatggtactgaagaaacaacgaaacaattcaactttgaagaagaagctgacacaatggaggatctatattacaatggagaagaagaagataaaactgatggagctgaagcatgtcctggacaatatttgatgagctgcaagaaagattacgacaatgtcataacggtttcagaaaagacagaagaaattgaagattgttatgagaataatgcaaggcatagactgaattacgaaaatgttatagcagcttgggataaacaagaatctcctagagatgtaaaaaacaacacaagtagcttccagctagttcctccagggatagaggagaagagagtaagaagtgagagagaagctagagtttggagatacagagataaaaggaagaatcgtttgttcgagaagaagataaggtacgaggttagaaaggttaacgccgacaaaaggccgagaatgaaaggtcgatttgtccgacgatctttagccatcgactcttag

SEQ ID NO: 2 - protein, 20 aa, Arabidopsis thaliana
AHYSPSLLALVFFIVPFFSL

SEQ ID NO: 3 - protein, 5 aa, Arabidopsis thaliana
DLSCS

SEQ ID NO: 4 - protein, 327 aa, Arabidopsis thaliana
PMTSHQNIKISEKIMISKYQEDVKQPRACELCLNKHAVWYCASDDAFLCHVCDESVHSANHVATKHERVCLRTNEISNDVRGGTTLTSVWHSGFRRKARTPRSRYEKKPQQKIDDERRREDPRVPEIGGEVMFFIPEANDDDMTSLVPEFEGFTEMGFFLSNHNGTEETTKQFNFEEEADTMEDLYYNGEEEDKTDGAEACPGQYLMSCKKDYDNVITVSEKTEEIEDCYENNARHRLNYENVIAAWDKQESPRDVKNNTSSFQLVPPGIEEKRVRSEREARVWRYRDKRKNRLFEKKIRYEVRKVNADKRPRMKGRFVRRSLAIDS